(19) United States
(12) Patent Application Publication
(10) Pub. No.: US 2025/0288474 A1
(43) Pub. Date: Sep. 18, 2025

(54) ABSORBENT ARTICLE WITH EMBOSSED SHAPING ELEMENT

(71) Applicant: ESSITY HYGIENE AND HEALTH AKTIEBOLAG, GÖTEBORG (SE)

(72) Inventors: Carin HÅKANSSON, Göteborg (SE); Angelica BURVALL, Göteborg (SE); Pia KALENTUN, Göteborg (SE); Moa HOLMSTRÖM, Malmö (SE)

(73) Assignee: ESSITY HYGIENE AND HEALTH AKTIEBOLAG, GÖTEBORG (SE)

(21) Appl. No.: 18/860,975

(22) PCT Filed: Jun. 22, 2022
(86) PCT No.: PCT/EP2022/066948

§ 371 Date: Oct. 28, 2024

(51) Int. Cl. A61F 13/538; A61F 13/15; A61F 13/472; A61F 13/511; A61F 13/53

(52) U.S. Cl. CPC A61F 13/538; A61F 13/472; A61F 13/51108; A61F 2013/15422; A61F 2013/530481

(57) ABSTRACT

An absorbent article in the form of a sanitary napkin or incontinence pad includes a fluid permeable topsheet, a backsheet and an absorbent core including a first and a second absorbent layer, the first absorbent layer being located between the topsheet and the second absorbent layer, and the second absorbent layer being located between the first absorbent layer and the backsheet. The absorbent core has a front part, a rear part and an intermediate part located between the front and rear parts. The first absorbent layer has a greater extension than the second absorbent layer in the plane of the article. The second absorbent layer includes a central embossed shaping element extending in the longitudinal direction and the second absorbent layer has a basis weight above 200 gsm and the first absorbent layer has a basis weight below 200 gsm.

### TECHNICAL FIELD

The present disclosure pertains to an absorbent article for a female user in the form of a sanitary napkin, incontinence pad or a liner having an absorbent core provided with embossed shaping elements to promote a good fit and leakage security.

### BACKGROUND

Sanitary napkins and incontinence pads to which this disclosure relates are typically intended to absorb body liquids, such as urine or menses. Such articles may be absorbent pads for heavier bladder leakage, thinner liners for light bladder leakage or sanitary napkins for menses.

Absorbent articles are generally provided with an absorbent core to receive and retain body liquids. For incontinence pads intended for heavier bladder leakage to function efficiently, the absorbent core must quickly acquire body liquids into the structure from the point of application and subsequently distribute the body liquids within and throughout the absorbent core to provide maximum leakage security. It is also desirable that such pads are comfortable and have a good fit, i.e., to be sized and configured to fit the limited space available in the crotch portion of the underwear while providing the wearer with a feeling of security. Additionally, there is desire that such articles are discreet, thin, and not visible even if the user wears tight fitting, which may be seen as being in contradiction with the above objects.

In view of the above, there is a need for an absorbent article for female users which does not leak, has good fit, is discreet to wear and in addition gives the user a visual perception of leakage security and good fit, enabling the user to feel secure and thereby enhancing the well-being of the user.

### SUMMARY

One or more of the above objects is achieved with an absorbent article for female users according to claim 1. Further advantages and advantageous features are disclosed in the following description and in the dependent claims.

An absorbent article for a female user in the form of a sanitary napkin, an incontinence pad or a liner is extending in a longitudinal direction L and a transverse direction T. The absorbent article has a first and a second longitudinal side edge and a front and a rear end edge and comprises a fluid permeable topsheet, a backsheet and an absorbent core.

The absorbent core comprising a first absorbent layer and a second absorbent layer, the first absorbent layer being located between the topsheet and the second absorbent layer and the second absorbent layer being located between the first absorbent layer and the backsheet. The absorbent core has a front part, a rear part and an intermediate part located between the front part and the rear part. The first absorbent layer has a greater extension than the second absorbent layer in the plane of the absorbent article. The second absorbent layer comprises a central embossed shaping element extending in the longitudinal direction of the absorbent article. The second absorbent layer has a basis weight above 200 gsm and the first absorbent layer has a basis weight below 200 gsm. To have a second absorbent layer with higher basis weight below a first absorbent layer, and that the upper first absorbent layer has a greater extension than the second absorbent layer makes the absorbent article relatively thin and also comfortable and discreet to wear. However, such pad may become difficult to shape in a predictable manner so as to conform to the body of the user and therefore a central embossed shaping element extending in the longitudinal direction enables an improved flexing and shaping of such pad.

The density in the second absorbent layer may be between 120 kg/m³ and 500 kg/m3 and the density in the first absorbent layer may be lower than the density in the second absorbent layer. Due to a relatively high density in the second absorbent layer, the core structure may be perceived as less flexible and less prone to shape in response to the user body. However, the central embossed shaping element enables an improved flexing and shaping of such pad. Furthermore, having a first absorbent layer with a greater extension above a second absorbent layer with higher density makes the absorbent article thinner in the areas outside the second core area, and hence, a more comfortable and discreet absorbent article to wear for the user.

The density of the second absorbent layer may be between 120 kg/m³ and 400 kg/m³, optionally between 135 kg/m³ and 350 kg/m³ and optionally between 150 kg/m³ and 350 kg/m³.

The first absorbent layer, in the plane of the absorbent article, may surround the second absorbent layer around its entire circumference, and this decrease the risk of the article to be perceived as stiff against the user when being worn.

The front part of the absorbent core may constitute 20-40% of the total longitudinal length of the absorbent core, or optionally 20-33% of the total longitudinal length of the absorbent core.

The greatest transversal width of the absorbent core may be in the front part. The transversal width of the absorbent core may be greater in the entire front part than in the entire intermediate and rear part.

The transversal width of the absorbent core in the intermediate and rear part may be equal or less than the transversal width of the absorbent core in the front part.

A transversal width between the front part and the intermediate part of the absorbent core may be narrower than the transversal width of the rest of the absorbent core. The transversal width of a transition between the front part of the absorbent core and the intermediate part of the absorbent core is defined W₈.

The central embossed shaping element may have an end point or a converging point coinciding or being adjacent with the transversal width W₈ between the front part and the intermediate part of the absorbent core. The distance in the longitudinal direction L between an end point or a converging point of the central embossed shaping element and the transversal width W₈ may not be greater than 10%, or 5% of the total length of the absorbent core.

The central embossed shaping element may extend along a longitudinal centreline Lc of the absorbent article. The distance in the transversal direction T between the central embossed shaping element and the longitudinal centreline may not be greater than 5% of the narrowest width of the absorbent core.

The second absorbent layer may comprise superabsorbent polymers in an amount of from 30 wt. %, optionally from 35 wt. % or optionally from 40 wt %. The second absorbent layer may comprise superabsorbent polymers within the range of from 30 wt %, 35 wt % or 40 wt. % to 80 wt. %. The second absorbent layer may also comprise superabsorbent polymers within the range of from 50 wt. % to 80 wt. %. Due to a higher content of superabsorbent polymers, the core structure may be perceived as less flexible and less prone to shape in response to the user body. Therefore, such article may become difficult to shape in a predictable manner. The central embossed shaping element however enables an improved flexing and shaping of such article without being experienced as stiff.

The second absorbent layer may be enclosed by a core wrap, such as for example a nonwoven wrap or a tissue wrap.

The second absorbent layer may comprise a first side cut along a first longitudinal side edge and a second side cut along a second longitudinal side edge. The first and the second side cuts may be arranged on either side of and aligned with the converging point or an end point of the central embossed shaping element. The side cuts cooperate with the central embossing element to shape the pad in a desired and predictable manner. The side cuts in the second core layer reduces the stiffness of the absorbent article and decrease the thickness of the absorbent article where the side cuts are located. The side cuts in combination with the central embossing element also make the absorbent core more comfortable for the user. The side cuts promote the absorbent core, during use of the pad to flex and to adopt a bowl like shape in the inlet zone in the front part. The first and the second side cuts may be arranged in the transition between the front part and the intermediate part of absorbent core.

The first side cut is defined between a first front deflection point and a first rear deflection point, and the second side cut is defined between a second front deflection point and a second rear deflection point. The central embossed shaping element in the longitudinal direction may end at the first front deflection point and the second front deflection point.

The distance between the front and the rear deflection points of the respective first and second side cuts may be within the range of from 20 to 80 mm.

A ratio of a width at the narrowest section of the second absorbent layer W₁₃ at the first and the second side cuts and a width of the incontinence pad W₁ at the same location may be from 0.5:1 to 0.8:1.

The transversal width between the front part and the intermediate part of the absorbent core may be narrower than the transversal width of the rest of the absorbent core and the 10 second absorbent layer may also have its narrowest transversal width between the front part and the intermediate part of the absorbent core. The transversal width of the second layer between the front part and the intermediate part of the absorbent core is defined W₁₃ and may be narrower than the transversal width W₈ of the absorbent core.

The front part, the rear part and the intermediate part of the absorbent core may be of substantially equal length.

The central embossed shaping element may comprise an I-shaped portion extending along at least part of the longitudinal centreline Lc of the second absorbent layer. The I-shaped portion in the longitudinal direction towards the front part, may have a front end point in the transition between the front part and the intermediate part of the second absorbent layer.

According to another embodiment the central embossed element has an I-shaped configuration and the front-end point of the I-shaped central embossed shaping element coincide in the transversal direction with the first front deflection point and the second front deflection point. The rear end point of the central embossed shaping element may be located closer to the rear end edge of the article than the first rear deflection point and the second rear deflection point.

The central embossed shaping element may comprise a V-shaped or modified V-shaped portion provided in the front part of the second absorbent layer, and the V-shaped or modified V-shaped portion including a V-shaped portion first leg and a V-shaped portion second leg converging in a converging point. During use, the absorbent article flex and 35 adopt a bowl like shape in the inlet zone in the front half where the V-shaped portion first and second legs may converge in the converging point. A narrow transversal width between the front part and the intermediate part of the absorbent core also interact with the converging point in the central embossed shaping element so as to promote the absorbent core to flex and to adopt a bowl like shape in the inlet zone. Furthermore, the side cuts in the second absorbent layer also interact with the converging point in the central embossed shaping element so as to promote the absorbent core to flex and to adopt a bowl like shape in the inlet zone.

The central embossed shaping element may be in the form of a Y-shaped or modified Y-shaped embossing comprising a V-shaped or modified V-shaped portion provided in the front part of the second absorbent layer and an I-shaped portion extending along at least part of the longitudinal centreline Lc of the absorbent core in at least part of the intermediate part of the second absorbent layer. According to this embodiment, the central embossed V-shaped or modified V-shaped embossing portion meets the I-shaped embossing part in the converging point of the V-shaped embossing part. For non-intersecting V-shaped embossing portions, for example a modified V-shaped embossing, the I-shaped embossing portion 15b meets the extension of the V-shaped portion first leg and the V-shaped portion second leg, and together form a Y-shaped or modified Y-shaped embossing.

The first absorbent layer may have a density between 20 kg/m³ and 120 kg/m³, optionally between 30 kg/m³ and 100 kg/m³.

The absorbent core may also comprise a fluid control layer arranged between the topsheet and the first absorbent layer. The second absorbent layer may have at least 50%, such as 75% or 100% higher density than each of the fluid control layer and the first absorbent layer.

The absorbent article may also comprise an outer embossed shaping element provided in the topsheet and/or the absorbent core. The outer embossed shaping element may comprise a first embossed line and a second embossed line, each extending in the longitudinal direction L and on a respective side of the central embossed shaping element. The central embossed shaping element and the outer embossed shaping element enables an improved flexing and shaping of such pad.

The outer embossed shaping element may be provided in the topsheet and/or the first absorbent layer. If the absorbent article comprising also a fluid control layer, the outer embossed shaping element may also be provided in the fluid control layer. According to one embodiment is the outer embossed shaping element provided in the topsheet, in a fluid control layer and in the first absorbent layer.

The first and the second embossed lines of the outer embossed element may converge in the direction from the front-end edge of the pad towards the rear end edge of the pad. The transversal distance between the first and the second embossed lines closest to the front-end edge of the pad may be 30-60 mm and the transversal distance between the first and the second embossed lines closest to the rear end edge of the pad may be 0.5-20 mm.

The outer embossed shaping element may extend up to or past a rear end point of the central embossed shaping element and/or extend up to or past a front-end point of the central embossed shaping element. The outer embossed shaping elements may be provided by continuous embossment portions, discontinuous embossment portions, for example a few discontinuous embossment portions or a multiply of small discontinuous embossment portions.

The central embossed shaping element and/or the outer embossed shaping element may comprise a continuous embossment portion or discontinuous embossment portions. If the central embossed shaping element comprises discontinuous embossment portions it may for example be a few discontinuous embossment portions or a multiply of small discontinuous embossment portions.

### BRIEF DESCRIPTION OF THE DRAWINGS

The present invention will be further explained hereinafter by means of non-limiting examples and with reference to the appended drawings wherein:

FIG. 1 shows a top plan view of an incontinence pad as disclosed herein;

FIG. 2 shows an exploded view of the incontinence pad in FIG. 1; and

FIG. 3 shows the incontinence pad from FIG. 1, flexed and shaped as during use.

### DETAILED DESCRIPTION

The invention will be described more closely below by reference to an exemplary embodiment. The invention may however be embodied in many different forms and should not be construed as limited to the embodiments set forth in the drawings and the description thereto.

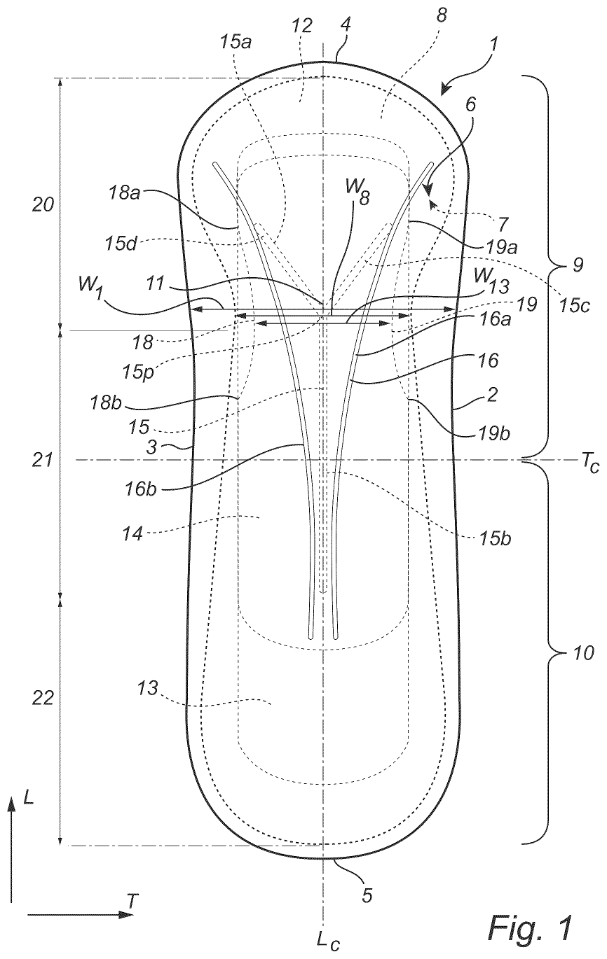

FIGS. 1 to 3 show an incontinence pad 1 according to the present disclosure. FIG. 1 schematically shows the incontinence pad 1 seen from the side that is intended to be facing towards a wearer's body when the pad 1 is being worn.

The incontinence pad 1 has a first and a second longitudinal side edge 2,3 and a front and a rear end edge 4,5. The pad 1 comprises a fluid permeable topsheet 6, a backsheet 7 and an absorbent core 8 being located between the topsheet 6 and the backsheet 7. The incontinence pad 1 extends in a longitudinal direction L with a longitudinal centerline Lc, and in a transverse direction T with a transverse centerline Tc and the absorbent core 8 is divided in a front half 9 and a rear half 10.

The absorbent core 8 comprises a first absorbent layer 12 and a second absorbent layer 13. The first absorbent layer 12 being located between the topsheet 6 and the second absorbent layer 13 and the second absorbent layer being located between the first absorbent layer and the backsheet 7. The second absorbent layer having a front part 20, a rear part 22 and an intermediate part 21 located between the front part 20 and the rear part 22. The first absorbent layer 12 has a greater extension than the second absorbent layer 13. The second absorbent layer 13 comprises a central embossed shaping element 15 extending in the longitudinal direction L of the incontinence pad 1 The second absorbent layer 13 has a basis weight above 200 gsm and the first absorbent layer 12 has a basis weight below 200 gsm. The second absorbent layer 13 is provided with a central embossed shaping element 15 extending in the longitudinal direction L so as to enable an improved flexing and shaping of the incontinence pad 1.

The density in the second absorbent layer is between 120 kg/m³ and 500 kg/m3 and the density in the first absorbent layer is lower than the density in the second absorbent layer. The density of the second absorbent layer may be between 120 kg/m³ and 400 kg/m³, optionally between 135 kg/m³ and 350 kg/m³ and optionally between 150 kg/m³ and 350 kg/m³.

The first absorbent layer 12 surrounds the second absorbent layer 13 around its entire circumference in the plane of the incontinence pad and the incontinence pad 1 is thinner in the areas outside the area of the second absorbent layer 13.

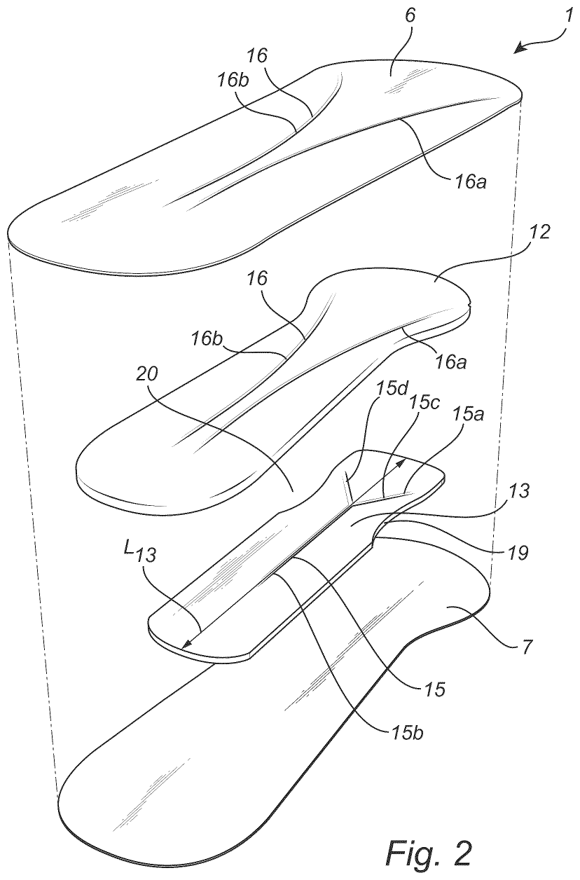

The front part 20 in the incontinence pad 1 in FIG. 1-2 constitutes ⅓ of the total longitudinal length of the absorbent core 8, the intermediate part 21 constitutes ⅓ of the total longitudinal length of the absorbent core 8 and the rear part 22 constitutes ⅓ of the total longitudinal length of the absorbent core 8. So, the front part 20, the rear part 22 and the intermediate part 21 of the absorbent core 8 of the incontinence pad 1 is of substantially equal length.

The greatest transversal width of the absorbent core 8 in the incontinence pad 1 in FIG. 1-2 is in the front part 20. The transversal width of the absorbent core 8 in the intermediate and rear part 21,22 is equal or less than the transversal width of the absorbent core 8 in the front part 20.

A transversal width of the second absorbent layer 13 between the front part 20 and the intermediate part 21 is defined W₁₃ and is narrower than the transversal width Wₑ of the absorbent core 8.

The central embossed shaping element 15 has a converging point 15p at the transversal width Wₑ between the front part and the intermediate part of the absorbent core. The distance in the longitudinal direction L between the converging point 15p of the central embossed shaping element 15 and the transversal width Wₑ may not be greater than 10%, or 5% of the total length of the second absorbent layer 13.

The converging point 15p of the central embossed shaping element 15 is at the longitudinal centreline Lc of the incontinence pad 1. The distance in the transversal direction T between the converging point 15p of the central embossed shaping element 15 and the longitudinal centreline Lc may not be greater than 5% of the narrowest width of the second absorbent layer 13.

The second absorbent layer 13 comprises superabsorbent polymers in an amount of from 30 wt. %, optionally from 35 wt. % or optionally from 40 wt %. The second absorbent layer may comprise superabsorbent polymers within the range of from 30 wt %, 35 wt % or 40 wt. % to 80 wt. %. The second absorbent layer may also comprise superabsorbent polymers within the range of from 50 wt. % to 80 wt. %. The second absorbent layer 13 is optionally wrapped in a core wrap.

The second absorbent layer 13 comprises a first side cut 18 along a first longitudinal side edge 2 and a second side cut 19 along a second longitudinal side edge 3 and the first and the second side cuts 19,20 are arranged on either side of and aligned with the converging point 15p of the central embossed shaping element 15. The side cuts cooperate with the central embossing element 15 to shape the incontinence pad 1 in a desired and predictable manner. The side cuts 19,20 in the second absorbent layer 13 reduces the stiffness of the incontinence pad 1 and decrease the thickness of the incontinence pad where the side cuts 19,20 are located. The side cuts 19,20 promote the second absorbent layer 13, during use of the incontinence pad 1 to flex and to adopt a bowl like shape in the inlet zone. The first and the second side cuts 19,20 are arranged in the transition between the front part 20 and the intermediate part 21 of absorbent core 8.

The first side cut 18 is defined between a first front deflection point 18a and a first rear deflection point 18b and the second side cut 19 is defined between a second front deflection point 19a and a second rear deflection point 19b. The central embossed shaping element 15 in the longitudinal direction ends at the first front deflection point 18a and a second front deflection point 19a. The distance between the front deflection points 18a, 19a and the rear deflection points 18b, 19b of the respective first and second side cuts may be within the range of from 20 to 80 mm.

A ratio of a width at the narrowest section of the second absorbent layer at the first and the second side cuts 19,20 and a width W₁ of the incontinence pad 1 at the same location is from 0.5:1 to 0.8:1.

The central embossed shaping element 15 comprises an I-shaped portion 15b extending along at least part of the longitudinal centreline Lc of the second absorbent layer 13. The I-shaped portion 15b in the longitudinal direction towards the front part 20, has a front-end point in the transition between the front part 20 and the intermediate part 21 of the absorbent core 8.

The central embossed shaping element 15 comprises a V-shaped portion 15a provided in the front part 20 of the second absorbent layer 13. The V-shaped or modified V-shaped portion 15a includes a V-shaped portion first leg 15c and a V-shaped portion second leg 15d converging in a converging point 15p. The central embossed shaping element 15 is in the form of a Y-shaped embossing comprising a V-shaped portion 15a provided in the front part 20 of the second absorbent layer and an I-shaped portion 15b extending along at least part of the longitudinal centreline Lc of the absorbent core 8 in at least part of the intermediate part 21 of the second absorbent layer 13. The central embossed V-shaped embossing part 15a meets the I-shaped embossing part 15b in the converging point 15p of the V-shaped embossing part 15a. For non-intersecting V-shaped embossing parts 15a, ie. a modified V-shaped embossing, the I-shaped embossing part 15b meets the extension of the V-shaped portion first leg 15c and the extension of the V-shaped portion second leg 15d, and together form a Y-shaped or modified Y-shaped embossing.

The first absorbent layer 12 has a density between 20 kg/m³ and 120 kg/m³, optionally between 30 kg/m³ and 100 kg/m³. The first absorbent layer 12 may be an airlaid nonwoven layer or a matformed absorbent layer. The absorbent core 8 may also comprise a fluid control layer arranged between the topsheet 6 and the first absorbent layer 12. A fluid control layer is adapted to quickly receive and temporarily store discharged liquid before it is absorbed by the first and second absorbent layers 12,13. A fluid control layer may be a fibrous layer comprising or consisting of synthetic fibers and may for example be airlaid nonwoven, spunlace nonwoven or high loft nonwoven.

The topsheet 6 may include or consist of fibrous nonwoven layer(s) being spunbonded, meltblown, carded, hydroentangled, wetlaid. Suitable nonwoven materials can be composed of natural fibers, such as woodpulp or cotton fibers, synthetic thermoplastic fibers, such as polyolefins, polyesters, polyamides and blends and combinations thereof or from mixtures of natural and synthetic fibers. The topsheet 6 may consist of non-absorbent fibers, such as thermoplastic fibers. The materials suited as topshet material should be soft and non-irritating to the skin and be readily penetrated by body fluid, such as menstrual fluid and urine.

The backsheet 7 may comprise or consist of a thin plastic film, e.g., a polyethylene or polypropylene film, a nonwoven material coated with a liquid impervious material, a hydrophobic nonwoven, which resist liquid penetration. Laminates of plastic films and nonwoven materials may also be used. The backsheet material can be breathable to allow vapor to escape from the absorbent structure, while still preventing liquids from passing through the backsheet material.

The absorbent core 8 constitutes the absorbent structure of the article which acquires, distributes and stores bodily fluids and comprises the first absorbent layer and the second absorbent layer. The first and second absorbent layers may be of any conventional kind. Examples of commonly occurring absorbent materials are cellulosic fluff pulp, tissue, highly absorbent polymers (so called superabsorbents), absorbent foam materials, absorbent nonwoven materials or the like. It is common to combine cellulosic fluff pulp with superabsorbent polymers in an absorbent core. Superabsorbent polymers are water-swellable, water-insoluble organic or inorganic materials capable of absorbing at least about times their own weight of an aqueous solution containing 0.9 weight percent of sodium chloride. Organic materials suitable for use as a superabsorbent material can include natural materials such as polysaccharides, polypeptides and the like, as well as synthetic materials such as synthetic hydrogel polymers. Such hydrogel polymers include, for example, alkali metal salts of polyacrylic acids, polyacrylamides, polyvinyl alcohol, polyacrylates, polyacrylamides, polyvinyl pyridines, and the like. Other suitable polymers include hydrolyzed acrylonitrile grafted starch, acrylic acid grafted starch, and isobutylene maleic anhydride copolymers and mixtures thereof. The hydrogel polymers are preferably lightly cross-linked to render the material substantially water insoluble. Preferred superabsorbent materials are further surface cross-linked so that the outer surface or shell of the superabsorbent particle, fibre, flake, sphere, etc. possesses a higher crosslink density than the inner portion of the superabsorbent. The superabsorbent materials may be in any form suitable for use in absorbent composites including particles, fibres, flakes, spheres, and the like. A high absorption capacity is provided using high amounts of superabsorbent material. Thin absorbent cores which are common in for example sanitary napkins, baby diapers and incontinence guards, often comprise a compressed, mixed or layered structure of cellulosic fluff pulp and superabsorbent polymers. The size and absorbent capacity of the absorbent core may be varied to suit different product types, such as smaller incontinence pads for day use or larger and longer ones for night-time use.

The incontinence pad 1 has an outer embossed shaping element 16 provided in the topsheet 6 and the first absorbent layer 12. The outer embossed shaping element has a first embossed line 16a and a second embossed line 16b, each extending in the longitudinal direction L and on a respective side of the central embossed shaping element 15. The central embossed shaping element 15 and the outer embossed shaping element 16 enables an improved flexing and shaping of the incontinence pad 1. The outer embossed shaping element 16 may also be provided in a fluid control layer. The first and the second embossed lines 16a,16b of the outer embossed element 16 converge in the direction from the front-end edge 4 of the incontinence pad 1 towards the rear end edge 5 of the incontinence pad 1. The transversal distance between the first and the second embossed lines 16a,16b closest to the front-end edge 4 of the incontinence pad 1 is 30-60 mm and the transversal distance between the first and the second embossed lines 16a,16b closest to the rear end edge 5 of the incontinence pad 1 is 0.5-20 mm.

The outer embossed shaping element 16 extends up to and past the rear end point of the central embossed shaping element 15 and also extends up to and past the front-end point of the central embossed shaping element 15. The central embossed shaping element 15 is in FIG. 1-2 one continuous embossment. If the central embossed shaping element comprises discontinuous embossment portions it may for example be a few discontinuous embossment portions or a multiply of small discontinuous embossment portions.

FIG. 2 shows an exploded view of the incontinence pad 1 in FIG. 1.

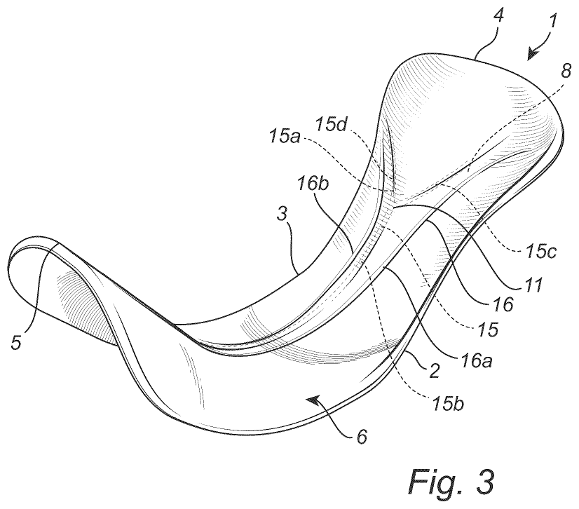

FIG. 3 illustrates the incontinence pad 1 from FIGS. 1 and 2 when being flexed and shaped as during use. The configuration of the absorbent core 8 comprising a central embossed shaping element 15 extending in the longitudinal direction L of the incontinence pad enables an improved flexing and shaping of such incontinence pad.

### Thickness Measurement

To determine the density of the first and the second absorbent layer is the thickness determined by means of a measuring foot with affixed load of 0.5 k Pa. The foot is lowered onto the absorbent layer at a rate of 13 mm/s. The thickness is read off at the digital thickness gauge/tester after 5 seconds when the measuring foot has touched the surface of the absorbent layer.

1. An absorbent article for a female user in the form of a sanitary napkin, incontinence pad or liner extending in a longitudinal direction and a transverse direction and having a first and a second longitudinal side edge, a front and a rear end edge and comprising a fluid permeable topsheet, a backsheet, and an absorbent core comprising a first absorbent layer and a second absorbent layer, the first absorbent layer being located between the topsheet and the second absorbent layer, and the second absorbent layer being located between the first absorbent layer and the backsheet, the absorbent core having a front part, a rear part and an intermediate part located between the front part and the rear part, the first absorbent layer has, in the plane of the absorbent article, a greater extension than the second absorbent layer wherein the second absorbent layer comprising a central embossed shaping element extending in the longitudinal direction of the absorbent article and that the second absorbent layer has a basis weight above 200 gsm and the first absorbent layer has a basis weight below 200 gsm.

2. The absorbent article according to claim 1, wherein the density in the second absorbent layer is between 120 kg/m³ and 500 kg/m³ and the density in the first absorbent layer is lower than the density in the second absorbent layer.

3. (canceled)

4. The absorbent article according to claim 1, wherein the first absorbent layer, in the plane of the absorbent article, is surrounding the second absorbent layer around its entire circumference.

5. The absorbent article according to claim 1, wherein the front part, the rear part and the intermediate part of the absorbent core are of substantially equal length.

6. The absorbent article according to claim 1, wherein a transversal width between the front part and the intermediate part of the absorbent core is narrower than the transversal width of the rest of the absorbent core.

7. The absorbent article according to claim 1, wherein the central embossed shaping element has an end point or a converging point coinciding with the transversal width between the front part and the intermediate part of the absorbent core.

8. The absorbent article according to claim 1, wherein the central embossed shaping element extends along a longitudinal centerline of the absorbent article.

9. The absorbent article according to claim 1, wherein the second absorbent layer comprises superabsorbent polymers in an amount of from 30 wt.

10. The absorbent article according to claim 1, wherein the second absorbent layer comprises a first side cut along a first longitudinal side edge and a second side cut along a second longitudinal side edge and the first and the second side cuts are arranged on either side of and aligned with the converging point or an end point of the central embossed shaping element.

11. The absorbent article according to claim 10, wherein the first side cut is defined between a first front deflection point and a first rear deflection point and the second side cut is defined between a second front deflection point and a second rear deflection point and wherein the central embossed shaping element in the longitudinal direction ends at the first front deflection point and the second front deflection point.

12. The absorbent article according to claim 10, wherein a ratio of a width at the narrowest section of the second absorbent layer at the first and the second side cuts and a width of the absorbent article at the same location is from 0.5:1 to 0.8:1.

13. The absorbent article according to claim 1, wherein the central embossed shaping element comprises an I-shaped portion extending along at least part of the longitudinal centreline of the second absorbent layer.

14. The absorbent article according to claim 1, wherein the central embossed shaping element comprises a V-shaped or modified V-shaped portion provided in the front part of the second absorbent layer, and the V-shaped or modified V-shaped portion includes a V-shaped portion first leg and a V-shaped portion second leg converging in a converging point.

15. The absorbent article according to claim 13, wherein the central embossed shaping element is in the form of a Y-shaped or modified Y-shaped embossing comprising a V-shaped or modified V-shaped portion provided in the front part of the second absorbent layer and an I-shaped portion extending along at least part of the longitudinal centreline of the absorbent core in at least part of the intermediate part of the second absorbent layer.

16. The absorbent article according to claim 1, wherein the first absorbent layer has a density between 20 kg/m³ and 120 kg/m³.

17. The absorbent article according to claim 16, wherein the absorbent core also comprises a fluid control layer arranged between the topsheet and the first absorbent layer.

18. The absorbent article according to claim 1, wherein the absorbent article also comprises an outer embossed shaping element provided in at least the first absorbent layer, and optionally also in the topsheet (6), the outer embossed shaping element comprising a first embossed portion and a second embossed portion, each extending in the longitudinal direction and on a respective side of the central embossed shaping element.

19. The absorbent article according to claim 18, wherein the outer embossed shaping element is provided in the topsheet and the first absorbent layer and optionally in a fluid control layer.

20. The absorbent article according to claim 18, wherein the first and the second embossed portions of the outer embossed element converge in the direction from the front-end edge of the article towards the rear end edge of the article.

21. The absorbent article according to claim 18, wherein the outer embossed shaping element extends up to or past a rear end point of the central embossed shaping element or extends up to or past a front-end point of the central embossed shaping element.
